(19) United States
(12) Patent Application Publication
(10) Pub. No.: US 2008/0076168 A1
(43) Pub. Date: Mar. 27, 2008

(54) Method of Carring Out a Chemical Reaction Between Reactants In a Reactor

(76) Inventors: Antonius Cornelis Petrus Maria Backx, Veldhoven (NL); Peter William Backx, Veldhoven (NL); Eric Anton Backx, Veldhoven (NL)

Correspondence Address: Richard S Roberts;Robert & Roberts LLP, Attorneys at Law, P O BOX 484, Princeton, NJ, 08542-0484, US

(21) Appl. No.: 11/663,220

(22) PCT Filed: Sep. 20, 2005
(86) PCT No.: PCT/NL05/00685

§ 371 Date: Jul. 6, 2007

(30) Foreign Application Priority Data
Sep. 21, 2004 (NL) 1027083

(51) Int. Cl. C12N 9/00

(52) U.S. Cl. 435/183

(57) ABSTRACT

A method of carrying out a chemical reaction between reactants in a reactor in the presence of a catalyst or an enzyme, wherein the process conditions on the catalytic surface of the catalyst are changed in relation to the process conditions as set in the reaction zone of the reactor, which change varies between two conditions, viz. a first condition, in which no catalytic activity is observed, and a second condition, in which such catalytic activity is actually observed.

Method of carrying out a chemical reaction between reactants in a reactor.

The present invention relates to a method of carrying out a chemical reaction between reactants in a reactor, which chemical reaction takes place in the presence of a catalyst or an enzyme.

Catalysts and enzymes play an important part in current processes in the petrochemical, chemical and biochemical process industries. The use of catalysts and/or enzymes makes it possible for (bio)chemical reactions to take place under relatively mild conditions. After all, if no catalyst or enzyme would be present, such reactions could only take place under severer conditions, for example higher pressures and/or temperatures. Catalysts and enzymes are furthermore known to boost specific reactions in a complex of possible reactions, so that catalysts and enzymes can also be regarded as agents that are capable of determining the selectivity in a process.

US patent application US 2004/0,097,371 relates to method of supplying the energy required for the reaction to catalyst molecules, making use of a support having thermal and electrical conductivity, wherein catalyst particles are dispersed therein or disposed thereon. The support can generate heat and activate the catalyst by using the thermal energy provided by the support, wherein electric energy is supplied to the conductive support. According to said patent, side reactions can be reduced and less energy is consumed by directing the heat through the support instead of heating the catalyst. However, a continuous supply of electric current to the catalyst is still required for activating the catalyst.

U.S. Pat. No. 5,730,845 discloses a method of producing an organo-nitrogen compound, comprising the step of selecting a catalyst susceptible to microwave radiation in a range of 1-20 GHz.

European patent application No. 0 135 357 relates to a reactor in which an endothermic catalytic process takes place, wherein the catalyst periodically undergoes a reactivation process.

From Dutch patent application NL 9301615 a catalyst present on a support is known, for example, which catalyst is used for the selective oxidation of sulfur-containing compounds to elemental sulfur, comprising at least one catalytically active material applied to a support material. In chemical processes, in which gases contaminated with sulfur compounds are released, it is desirable that the hydrogen sulfide be removed from said gases. Thus the Claus process is known, wherein the hydrogen sulfide is not quantitatively converted into elemental sulfur, mainly as a result of the equilibrium reaction. The residual gas obtained is passed, together with hydrogen, over a cobalt oxide/molybdenum oxide catalyst applied to Al₂O₃ as a support, whereby the SO₂ that is present is catalytically reduced to H₂S. A suitable selection of the process conditions and the catalyst components leads to a substantially complete conversion of hydrogen sulfide.

In principle, four parameters are relevant for determining and influencing the course of a (bio)chemical reaction process, viz. the concentration of the reactants, the pressure, the temperature and the residence time. By having the process take place in the presence of a catalyst and enzymes, the intended reactions will take place under milder process conditions. Generally, the average reactor conditions in the reaction zone will therefore be set so that catalysts and enzymes are utilised optimally for achieving the desired activity, selectivity and conversion. In practice this means, for example, that the entire reactor contents are maintained under optimum conditions for the reaction with the catalyst or the enzyme, which involves high energy costs. It will be understood that realising and maintaining such conditions for the entire reactor contents or a significant part thereof will lead to high energy costs. In addition, such a method reduces the possibilities of quickly and/or locally correcting or changing the course of the process, as the intended correction requires a manipulation of one or more main energy and/or main mass flows in such a situation. Generally, the possibilities of interfering with these flows are limited in amplitude and adjusting rate. Since they affect the entire reactor contents, or a significant part thereof, the manipulations of these main flows furthermore take effect relatively slowly, which is undesirable in practice.

The object of the present invention is to provide a method for carrying out a chemical reaction between reactants in a reactor, wherein the catalyst or the enzyme itself is used as a means for steering and controlling the course of the reactions that take place on the catalyst or the enzyme.

Another object of the present invention is to provide a method of carrying out a chemical reaction between reactants in a reactor, wherein process conditions that directly influence the course of the reaction with a view to obtaining an intended selectivity, activity and conversion are realised in the direct vicinity of the catalyst or the enzyme or on the active part thereof.

The invention as referred to in the introduction is characterized in that the process conditions on the catalytic surface of the catalyst are changed in relation to the process conditions as set in the reaction zone of the reactor, which change takes place between two conditions, viz. a first condition, in which no catalytic activity is observed, and a second condition, in which such catalytic activity is actually observed.

Using the present method, the catalyst or the enzyme is used as a means for steering and controlling the course of the chemical reactions, wherein the reaction can be steered to a situation in which no reaction takes place and a situation in which a reaction does take place, which situation may also comprise maximisation of the reaction, or to a condition between said situations, which is done by locally changing the process conditions on the active surface of the catalyst in relation to the nominal process conditions as set in the reaction zone in the reactor. The term “change” as used herein is to be understood to mean the active and dynamic steering between the first and the second condition, but also the adjustment of the process conditions on the catalytic surface or the active part thereof to the first or the second condition. The terms “catalyst” and “enzyme” are regarded as synonyms herein, and consequently the term “catalyst” can also be read as “enzyme” in all cases.

The present invention relates to adaptations to the catalyst, the catalyst/enzyme support material or the enzyme such that conditions in the direct vicinity of the active catalyst/enzyme sites can be changed dynamically and continuously for the purpose of manipulating the course of the reactions as desired in the range between no significant reaction—the adjusted nominal reactor condition—and a maximum reaction—with a fully activated catalyst/enzyme—without any adaptation of the average reactor conditions being required.

If the process conditions on the catalytic surface are set so that the catalyst or the enzyme does not enable the chemical reactions to take place, or only at a slow rate, under the prevailing process conditions in the reaction zone in the reactor, the catalyst will hardly be active. According to the present invention, it is possible to change the process conditions that locally prevail on the catalyst dynamically into specific process conditions, such that the course of the reaction that takes place on the catalytic surface is directly influenced and consequently the intended object can be achieved, viz. activity, selectivity, conversion and reaction rate, without having to adapt the overall process conditions in the reactor. It is possible to effect such a change once, so that the second situation prevails, but it can also be carried out repeatedly, viz. dynamically, as a function of time, so that the conditions that prevail on the catalytic surface are constantly adapted, for example in order to maximise one or more of the following factors: activity, selectivity, conversion and reaction rate. Since the present inventor has proceeded from the fact that chemical reactions take place on the catalytic surface, the present invention is directed at influencing only the conditions that prevail on said catalytic surface without at the same time influencing the conditions at locations far removed from the catalytic surface. Such an approach makes it possible to steer the course of the reaction in an active and energetically advantageous manner.

According to the present invention, said changing of the process conditions on the catalytic surface of the catalyst is effected by actively steering the conditions that prevail on the active part of the catalyst in relation to the conditions that prevail in the reactor. The changes or manipulations that apply according to the present invention may consist of simultaneous or sequential manipulations of one or more of the following mechanisms:
changing the temperature of the catalyst on the catalytic surface,
changing the surface structure properties or the structure properties of the active part of the catalyst,
depositing or removing one or more components on/from the catalytic surface,
changing the accessibility of reactants to the catalytic surface,
changing the absorption behaviour of the product(s) formed on the catalytic surface,
changing the temperature of the support material on which the catalytic surface is present, and
applying an electromagnetic field around the catalytic surface.

The essence of the present invention is in particular the special use of the catalyst and the enzyme, viz. for steering and controlling the reactions that take place on the catalytic surface in or between two conditions, viz. a first condition in which no catalytic activity is observed and a second condition in which catalytic activity is actually observed. According to the present invention, it is thus possible to adjust and vary the current process conditions that prevail on the catalytic surface between the above two conditions.

The present invention relates to a mechanism that makes it possible, for example, to directly or indirectly increase the local temperature of the active catalyst sites for a particular period of time, so that the “activation energy” thresholds that are necessary for the reaction processes are exceeded during said period. Said temporary increasing of the local temperature of the active catalyst/enzyme sites can be realised, for example, by locally producing heat in the direct vicinity of the active sites, which heat production can be activated/deactivated. Said increasing of the temperature can take place directly, by periodic, dynamic excitation of the catalyst/enzyme molecules, or indirectly, by periodically heating the vicinity of the active catalyst/enzyme sites locally for short intervals.

According to another embodiment, a mechanism is provided which locally changes the accessibility condition of active catalyst/enzyme sites for one or more of the reactants from non-accessible to accessible for a particular period of time. This can be realised, for example, by using catalyst/enzyme support materials whose form—for example its channel or pore dimensions or, for example, its surface structure—is changed for a particular period of time through manipulation of the field strength of an electromagnetic field.

Furthermore it is possible to use a mechanism that changes the orientation of one or more or reactants for a particular period of time, from an orientation in which the molecules are not correctly oriented for binding to the active catalyst/enzyme sites to an orientation in which such binding is possible. This can be realised, for example, by orienting molecules of reactants that form an electric dipole by means of an applied electric field, or by orienting molecules which form a magnetic dipole themselves or which are magnetisable by means of an applied magnetic field. These fields can be dynamically turned on and off for activating/deactivating the course of the reaction process.

According to yet another embodiment, the accessibility of active catalyst/enzyme sites is locally changed for a particular period of time, in such a manner that the energy level at which one or more of the relevant reactants reach the active catalyst/enzyme sites can be manipulated between a condition in which sufficient energy remains for binding to the active catalyst/enzyme sites and a condition in which this is not the case. This can be realised, for example, by locally withdrawing energy from the reactants in question in the deactivated condition, which is done by changing the local surface structure of the catalyst or the enzyme, wherein energy is withdrawn from the relevant reactants through collisions with molecules of non-active catalyst/enzyme sites.

Yet another embodiment comprises a mechanism that locally changes the desorption of resultant reaction products for a particular period of time between an active condition and a non-active condition so that the resulting products can readily leave the active catalyst/enzyme site in the active condition and are unable to leave said active catalyst/enzyme site in the non-active condition. This can be realised, for example, by using catalyst/enzyme support materials whose form—for example its channel or pore dimensions or, for example, its surface structure—is changed for a particular period of time through manipulation of the field strength of an electromagnetic field.

Essential for the present invention is the fact that the mechanisms as described above can be used for dynamically manipulating the course of reactions in the full range between two conditions as required:
A first condition—the non-active condition—in which no significant reaction takes place
A second condition the active condition—in which reactions take place at a maximum rate

The manipulation of the course of the reactions takes place by changing local conditions, which can be done dynamically. Because only local conditions need to be manipulated, it is possible to effect relatively rapid dynamic responses, In many cases, these quick responses are required in order to be able to steer the current reaction process directly.

The present invention will be explained in more detail hereinafter by means of two examples, in which connection it should be noted, however, that the present invention is by no means limited to such special examples.

### EXAMPLE 1

A 10 vol. % solution of hydrogen peroxide was charged to a reactor vessel present in a stirred bath of melting ice. A manganese oxide catalyst was used, which manganese oxide catalyst was present on a support of aluminium foil. The support could be actively heated by means of a heating element, whilst also the surface temperature could be measured by means of an NTC resistor. No decomposition of hydrogen peroxide into water and oxygen could be detected on the catalyst surface when the average reaction temperature of the hydrogen peroxide solution was about 0° C. The local heating of the catalyst surface by means of the heating element in the hydrogen peroxide solution, which hydrogen peroxide solution was still present in the ice bath, so that the temperature thereof was 0° C., led to a significant increase of the reaction rate. The increase in the reaction rate was readily detectable in that gas bubbles, in particular oxygen bubbles, formed on the catalyst surface. When the temperature of the catalyst surface was further increased to 30° C. or higher, a distinct increase in the amount of gas bubbles and a very rapid formation of gas bubbles was observed, in which connection it should be noted that the average temperature of the hydrogen peroxide solution was still maintained at 0° C. by means of the bath of melting ice. The formation of gas bubbles directly decreased when the local heating of the catalyst surface was stopped, which indicates that no catalytic activity took place on the catalytic surface. When subsequently the catalyst surface was heated anew, gas bubbles were observed to form on the catalytic surface again, which formation of gas bubbles stopped as soon as the heating of the catalytic surface by means of the heating element was terminated, which led to the catalyst surface assuming the temperature of the hydrogen peroxide solution, viz. a temperature corresponding to that of the ice bath. The amount of energy supplied for heating the catalyst surface is much too low to raise the temperature of a significant part of the reactor contents to the required reaction temperature. According to the present invention, the temperature required for the reaction was only realised very locally on the active part of the catalyst surface, which means an enormous saving in energy costs. Besides an improved energy household, the present invention in particular provides a possibility of quickly, precisely and directly influencing the course of processes, which results in a significant improvement as regards the controllability of these processes. This is achieved in that the influencing of said local conditions can be realised very quickly whilst using low energy levels.

The example as discussed above shows that changing the process conditions on the catalytic surface between a first condition, in which no catalytic activity is observed, viz. when the temperature of the catalytic surface is about 0° C., and a second condition, in which catalytic activity is observed, viz. when the temperature on the catalytic surface is increased to a value higher than that of the ice bath, in particular a value of at least 30° C., the catalyst can be used as an active agent for steering and controlling the course of the reactions that take place on the catalytic surface.

### EXAMPLE 2

Two identical silicon/silicon dioxide slices prepared for polymerisation are placed in a laboratory dish reactor for producing polyethylene on an activated chromium catalyst. The preparation of the slices concerns the application of a chromium compound to the thin (<1 μm) silicon dioxide surface layer of the slices by means of a spin-coating process. In the reactor, said layer is converted into an active chromium catalyst by means of a calcination process. The calcination process requires heating of the slices to a temperature above 550° C. for more than an hour by means of the electrical heating elements that are present in the reactor. The reactor atmosphere is conditioned to the required high purity level by vacuum cleaning prior to the start of the activation of the catalyst, after which the gas flows required for the reactions are passed over the slices. After such activation, the two slices comprise active chromium catalyst on the surface. Careful purification of the gas flows being supplied to the reactor ensures that the reactor atmosphere contains only very low concentrations of components that are harmful to the activated chromium catalyst.

Two identical silicon slices are mounted in the reactor. One of said slices is activated and deactivated by means of control signals. The other slice is not actively controlled. For the rest, the conditions in the reactor are completely identical for the two slices.

A platinum heating element (nominal resistance 235 ohm) and a tantalum temperature sensor (nominal resistance 2800 ohm) are vapour-deposited on the surface of the silicon slices that are mounted in the reactor. These control and measuring elements and the silicon surface are covered with a very thin layer of silicon dioxide (layer thickness <1 μm). The active chromium is chemically bound to the silicon dioxide. After the initialisation reactions, the two slices comprise active chromium catalyst sites on the surface. The nominal reactor conditions are set at values, T=60° C. and p=2 atm, that ensure that no significant reaction will take place on the active catalyst sites. Only one of the two slices is activated through intermittent local heating of the active catalyst sites. The other slice is continuously maintained at the nominal reactor conditions.

The reaction is allowed to continue for a period of 12 hours. The catalyst control results in the production of polyethylene on the slice that is actively controlled. Practically no polyethylene is produced on the slice that is not actively controlled.

The above-described mechanism for catalyst/enzyme activation/deactivation can be used on practically all catalytic and enzymatic processes. As a result of the active control of the catalyst/enzyme, conditions in which activation thresholds for the reactions are exceeded are locally realised on the active catalyst/enzyme sites during the (brief) activation. As a result of said activation, the reactions proceed as intended. Hardly any reaction takes place during the non-activated periods, because the bulk conditions in the reactor are set so that no significant reaction takes place. Because it is possible to realise large differences between the bulk conditions as set and the current, locally controlled conditions on the active catalyst/enzyme sites, it is possible to have strongly exothermal reactions take place in an intrinsically safe manner. The bulk conditions are to that end set so that significantly more power than the power that is produced by the reaction is locally discharged in the non-activated condition of the catalyst. The process will at all times stop in the non-activated condition of the catalyst/enzyme in that case and is intrinsically safe.

The large current difference between the bulk conditions and active catalyst/enzyme site conditions in the activated condition makes it possible to operate processes that are at present not possible in large-scale reactors. An example of this is the production of polymers or other materials at temperature conditions in which the product would undergo a phase transition—for example from solid to liquid, from liquid to gas or from solid to gas—if the bulk conditions would be equal to the brief local conditions in the activated condition. The production of polyethylene at temperatures well above about 130° C. is an example of this. The large current temperature gradient between reactor bulk (T<<80° C.) and active catalyst sites (T>130° C.) prevents this phase transition from taking place. Another possible example of this is the enzymatic modification of starch. The starch can be modified without decomposition or denaturalisation of the starch by realising the relatively low temperature bulk conditions (T<50° C.) and by brief, periodic realisation of the high temperature required for modification of the starch on the active enzyme sites by means of active enzyme control.

Different reactions can be made to take place simultaneously, intermittently or sequentially in one reactor by selectively controlling the active catalyst/enzyme sites. To that end, several catalysts/enzymes to be separately activated may be charged to the reactor, if necessary. By separately controlling the active catalyst/enzyme sites, the desired reactions can be activated and deactivated in the same reactor at the desired moments. This mechanism can be used, for example, for producing so-called “multimodal” polymers in one reactor.

1. A method comprising carrying out a chemical reaction between reactants in a reactor in the presence of a catalytic surface of a catalyst or an enzyme, wherein process conditions on the catalytic surface of the catalyst or enzyme are changed compared to process conditions as set in a reaction zone of the reactor, which changed process conditions vary between two conditions, a first condition, in which no catalytic activity or enzymatic activity is observed, and a second condition, in which catalytic activity or enzymatic activity is observed.

2. The A method according to claim 1, wherein the process conditions are dynamically changed for of manipulating the course of the chemical reaction in the range between no significant reaction and a maximum reaction without adapting average reactor conditions.

3. The A method according to claim 1 wherein said changed process conditions comprises the changing of the temperature of the catalytic surface.

4. The method according to claim 1 wherein said hanged process conditions comprises the changing of the surface structure of the catalyst or enzyme, or an active part thereof.

5. The method according to claim 1 wherein said changed process conditions comprises the deposition or the removal of one or more components on or from the catalytic surface.

6. The A method according to claim 1 wherein said changed process conditions comprises the changing of the accessibility of reactants to the catalytic surface.

7. The method according to claim 1 wherein said changed process conditions comprises the changing of the sorption behaviour of the product formed on the catalytic surface.

8. The method according to claim 3 wherein said changing of the temperature takes place indirectly, by changing the temperature of a support material on which the catalytic surface is present.

9. The A method according to claim 4, wherein said changing of the surface structure comprises the application of an electromagnetic field around the catalytic surface.

10. The A method according to claim 1 wherein the process conditions on the catalytic surface are varied between the first condition and the second condition during the time the reactants are present in the reactor.

11. The method according to claim 2 wherein said changed process conditions comprises the changing of the temperature of the catalytic surface.

12. The method according to claim 2 wherein said changed process conditions comprises the changing of the surface structure of the catalyst or enzyme, or an active part thereof.

13. The method according to claim 2 wherein said changed process conditions comprises the deposition or the removal of one or more components on or from the catalytic surface.

14. The method according to claim 2 wherein said changed process conditions comprises the changing of the accessibility of reactants to the catalytic surface.

15. The method according to claim 2 wherein said changed process conditions comprises the changing of the sorption behaviour of the product formed on the catalytic surface.

16. The method according to claim 2 wherein the process conditions on the catalytic surface are varied between the first condition and the second condition during the time the reactants are present in the reactor.

17. The method according to claim 3 wherein the process conditions on the catalytic surface are varied between the first condition and the second condition during the time the reactants are present in the reactor.

18. The method according to claim 4 wherein the process conditions on the catalytic surface are varied between the first condition and the second condition during the time the reactants are present in the reactor.

19. The method according to claim 5 wherein the process conditions on the catalytic surface are varied between the first condition and the second condition during the time the reactants are present in the reactor.

20. The method according to claim 6 wherein the process conditions on the catalytic surface are varied between the first condition and the second condition during the time the reactants are present in the reactor.
